(19) United States
(12) Patent Application Publication
(10) Pub. No.: US 2004/0044345 A1
(43) Pub. Date: Mar. 4, 2004

(54) Shallow penetration bone screw

(76) Inventors: Richard Marshal DeMoss, Berthoud, CO (US); Travis Rian Andrews, Boulder, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 10/231,820

(22) Filed: Aug. 28, 2002

(51) Int. Cl. A61B 17/86

(52) U.S. Cl. 606/73

(57) ABSTRACT

The invention is directed to a self-drilling self-tapping bone screw that is especially useful in thin bone anchoring. That is, the bone screw provides an enhanced holding force for thin bone applications, such as cranial applications. In one aspect, the bone screw contains a retaining thread that expands in at least one dimension over a majority of its length. In this regard, the retaining thread makes original bone-to-screw contact over a majority of its entire length during insertion, thereby increasing the holding force between the bone and the screw. In a second aspect, the bone screw contains a generally tapered body section that expands from a first diameter (which may be zero) to a second greater diameter near the screw head. Accordingly, a thread on the outside surface of the tapered body section increases in diameter over its length to provide an increased holding force.

### FIELD OF THE INVENTION

The present invention relates to bone screws utilized in medical procedures. More particularly, the present invention is directed towards a self-drilling, self-tapping shallow penetration bone screw utilized to securely affix implant hardware to a bone surface. The bone screw is particularly apt for attaching implantable devices to thin bones including the attachment of implantable hearing aid devices to one of the cranial bones (e.g., the temporal bone).

### BACKGROUND

In many medical procedures, it is desirable to utilize one or more bone screws to either directly fasten two bone fragments together or to affix a thin “mender” plate to two or more bone fragments to aid in the healing process. Additionally, bone screws are utilized to affix any of a number of implantable devices to bone surfaces. In order to facilitate placement, some bone screws are both self-drilling and self-tapping. That is, these bone screws do not require a pilot hole be drilled prior to their insertion. These self-drilling bone screws typically utilize some sort of cutter on their tip that removes a portion of the bone and allows for threads on the screw to “tap” into the bone as the screw is inserted (i.e., turned). Generally, self-tapping screws are able to provide better bone-to-screw contact (i.e., greater gripping force) than screws that require a predrilled pilot hole.

In some instances, such as affixing implantable devices to cranial bones, it is desirable to securely affix an implantable device to the target cranial bone while minimally intruding into the bone. As will be appreciated, cranial bones generally have a thickness of between about 4 mm and about 6 mm requiring bone screws utilized to attach implantable devices to provide an adequate gripping force over a relatively short distance. Therefore, in cranial applications, as well as other thin bone applications, a short geometry bone screw needs to provide a desired gripping force over a minimal insertion distance. Further, during bone screw insertion in thin bone applications, care must be taken to prevent “stripping” the threads tapped into the bone by the screw, which typically ruins an insertion point and requires repositioning of an implantable device.

### SUMMARY

It is a primary objective of the present invention to provide a bone screw that maximizes its holding force over a minimal insertion depth within a bone;

A secondary objective of the present invention is to provide a bone screw that attains an increased surface contact between a bone and screw thread;

Another objective of the present invention is to provide a self-drilling bone screw that removes a minimal amount of bone;

Another objective of the present invention is to provide a self-tapping bone screw that is resistant to bone thread stripping;

A related objective of the present invention is to provide a self-drilling, self-tapping bone anchor for use in surgical procedures.

One or more of the above noted objectives, as well as additional advantages, are provided by the self-tapping, self-drilling bone screw of the present invention that provides increased holding force over a minimal bone insertion distance. Generally, this bone screw comprises three sections: a tip section having a point and cutter to initiate insertion of the screw into a bone, a body section containing a helical retaining thread formed thereon for tapping a thread into the patient's bone, and a head section for use in affixing a prosthetic bracket to a bone surface as well as receiving a rotating force for insertion of the bone screw.

According to a first aspect of the present invention, a self-drilling, self-tapping bone screw is provided having a continuous helical thread formed on the outside surface of the body section and extending over at least a portion of the screw between the tip section and the head section. The outside diameter of the helical thread, as measured from the centerline axis of the screw expands, beginning near the tip, over a majority of the its length allowing a majority of the thread to tap into previously undisturbed bone during insertion of the screw. That is, unlike screws having a series of uniform threads, some of which may pass through a thread cut into a bone by a previous like-sized thread, the increasing outside thread diameter ensures that the a majority of the helical thread is making original screw-to-bone contact. This original contact helps eliminate thread wear caused by like-sized threads on the screw passing through a like-sized thread previously tapped into the bone and thereby provides a screw having increased gripping force. In one embodiment, the outside diameter of the thread expands over at least seventy-five percent of its length to further enhance the gripping force of the screw.

Additions and various refinements of the noted features exist. These refinements and additional features may be provided separately or in any combination. For instance, the bone screw may be constructed of any material that imparts desired bio-compatibility and mechanical properties to allow the bone screw to be permanently inserted within a bone while providing adequate retaining strength to maintain attachment of an implantable device to the bone. These materials may include, without limitation, composite materials, metals, and/or metal alloys. It has been found that titanium and titanium alloys typically provide the best combination of bio-compatibility and mechanical properties.

As noted in the first aspect, the helical thread of the inventive bone screw may expand in diameter over a majority of its length, or a first portion of the body section. In one embodiment at least a second dimension of helical thread expands over at least a second portion of the body section. This secondary expansion allows additional portions of the helical thread to provide enhanced contact with previously undisturbed bone during insertion of the screw. That is, the helical thread may expand in diameter over a first portion of the screw body as measured from the tip of the screw and expand in another dimension over a second portion of the screw body.

For example, most threads are generally defined by four separate elements: a leading flank, a trailing flank, a top or crest surface of the thread, and a root surface between successive threads. In this regard, along the first portion of the screw, the outside diameter of the helical thread may be continuously expanding while along a second portion of the helical thread, which may extend beyond the first portion as measured from the screw tip, the dimensions of at least one of the above noted elements may be expanding. In this regard, additional subsequent portions of the helical thread may continue to increase in at least one dimension in relation to previous thread portions allowing more of the helical thread to achieve original screw-to-bone contact. For example, the outside diameter of the helical thread may expand over a first portion of the screw body at the end of which the outside thread diameter may become a constant value. Accordingly, a dimension of any of the above noted elements, such as the diameter of the root surface separating the successive helical threads, the angle of one of the flanks, and/or the crest width, may expand throughout a second portion of the screw where the outside diameter of the helical thread is a constant value. This allows the second portion of the helical thread to continue expanding and to provide a bone screw having further enhanced gripping strength. Further, it will be appreciated that the first and second portions of the thread may be separate, abutting, or, they may completely or partially overlap. Furthermore, one or more of the thread dimensions may continuously expand over the entire length of the thread. For example, an outside diameter and/or a root diameter of the thread may continuously expand over the entire helical thread length.

Generally, the helical thread will have a constant pitch (i.e., distance between successive thread coils on the outside surface of the body section) so that subsequent portions of the screw thread may expand in the previously tapped bone thread. That is, the continually expanding screw thread will continue to expand, for example outward, into the bone thread but will not apply linear forces between successive bone threads that may result in bone separation, bone powdering, or other damage to the bone structure. The screw threads may be formed having leading and trailing flanks of any angle relative to the screw's central axis. However, it is preferable that the surface area of the front flank is increased in relation to the trailing flank to provide increased surface area for contact with the bone. That is, the leading flank may have a small angle (e.g. less than 45°) relative to the central axis of the screw so that it forms a long sloping surface. In this preferred embodiment, the trailing flank has a more perpendicular flank angle with regards to the central axis to provide increased resistance to axial extraction forces. In one preferred embodiment, the trailing flank angle is at least about 85° as measured from the central axis of the screw.

In order to provide a continuous helical thread that expands in diameter over a majority of its length, the body section of the screw may be tapered over a portion of its length between the tip and head of the screw. For example, a majority of the body section may be tapered such that the body is substantially conical. As will be appreciated, by forming a thread on the conical surface, the outer diameter of the thread may continuously expand as the thread winds around the conical surface.

The tip section of the screw has a cutter that enables the screw to remove a portion of the bone as it is inserted therein. Various configurations exist for screw tip cutters, any of which may be utilized with the present invention. However, in one embodiment, a recessed cutting flute is formed into the screw tip section. Generally, this cutting flute will comprise a recess formed at, or near, the point of the tip section while extending along a portion of the tip section of the screw. This recess may be formed in any way that provides an adequate edge for cutting into the bone as the screw is rotated. For example, the recess may be formed of two substantially planar surfaces that intersect at a right angle. Regardless of the exact configuration of the recess planes, it is preferable that the intersection of these planes be aligned with the screw's central axis at the tip. This central alignment at the tip reduces wobbling during insertion and allows the screw to be inserted substantially at the point where the screw is placed on a bone surface.

During screw insertion, the cutting edge of the flute scrapes away a portion of the bone which is in turn deposited into the recess. In one flute embodiment, the fluted recess is configured to expel this removed bone matter as additional bone matter accumulates. In this regard, one surface of the recess may be curved and rise to a root surface between two successive coils of the helical thread where the bone matter may be expelled.

In a further embodiment, the thread begins at a point along the screw somewhere behind the tip section and its cutter. As will be appreciated, the beginning or leading point of the thread is formed on the outside surface of the body section and is therefore somewhat offset from the central axis of the screw. During initial screw insertion this leading point may cause a shifting force to be applied to the screw. By having a tip section and cutter that are formed before the beginning of the thread (i.e., free of the helical thread), a portion of the screw may be inserted into the bone prior to the leading point of the thread contacting the bone surface and prevent this slightly offset thread point from shifting the screw. That is, the tip section may act as a spindle or pin in the bone that prevents the screw from moving laterally relative to the bone's surface during initiation of screw thread insertion.

According to a second aspect of the present invention, a self-drilling, self-tapping bone screw is provided that contains a screw head and a body section extending from the screw head and terminating in a point. The body section contains a helical thread formed along at least a portion of the length of its outside surface as well as a cutting flute associated with the point. In this second embodiment of the present invention, the body section is tapered from a first diameter beginning at or near the point to a second diameter ending near the screw head, wherein the second diameter is greater than the first diameter. The entire length of the body section need not be tapered; however, to allow for an enhanced gripping force, at least 50 percent of the body section and, more preferably, at least 75 percent of the body section will be tapered. That is, the body of the screw is generally tapered but may contain a portion having a constant outside diameter.

The tapered body section may comprise any shape that expands from a first diameter (which may be at the point and have a zero diameter) to a second larger diameter. However, in one embodiment, the tapered body section defines a circular cone that expands linearly from a point to the second diameter. This circular cone contain an included angle at the point of less than about 45°. As will be appreciated, this included angle will control the overall length of the tapered section of the screw for a given second diameter. For example, if the screw expands from a point to having an second diameter of 1 mm. near the screw head, a tapered section having an included angle of 30° will be longer than a tapered section having an included angle of 45°. For most applications, it has been found that an included angle of about 32° provides a screw with sufficient internal structure to be inserted into a bone while providing adequate length to effectively thread into and grip the bone.

The helical thread formed on the outside surface of the body section will have an expanding outside diameter over the entire length of the tapered section. Preferably, at least one additional thread dimension will expand over substantially the entire length of the body section including tapered and non-tapered body sections. As in the above first aspect of the present invention, the expanding thread elements may include any or all of the following non-inclusive list: the root surface diameter measured from the centerline axis of the bone screw, a crest width of the helical retaining thread, and/or the angles of the leading and trailing flanks of the thread. Furthermore, the separate elements may expand over separate portions of the screw to combinatively provide continued expansion.

According to another aspect of the present invention, a self-drilling, self-tapping bone anchor is provided. This bone anchor contains a head for receiving insertion torque that inserts the anchor into a bone and a body section extending from the head and terminating in a point that includes a cutter for initiating insertion of the anchor into a bone. Along the body section, a continuous helical thread is formed having at least one dimension that expands along a majority of the thread to allow that thread to continuously tap into a bone during the insertion of the anchor body. Finally, the self-tapping bone anchor contains a retention element associated with the head for selectively receiving and retaining a surgical securing device.

The bone anchor retention element may include any structural formation that is capable of retaining a surgical securing device. For example, the retention element may be a lip formed by the outside surface of the head. As will be appreciated, a surgical securing device such as a suture or a wire may be wound around the body section and thereby trapped between this lip and the bone surface. Alternatively, the head of the anchor may contain an aperture through which a surgical securing device such as a wire or suture may be routed.

In a related aspect of the present invention a method for inserting a self-drilling, self-tapping bone screw into a bone is provided. The method comprises positioning the tip of the bone screw at a desired location on a bone surface. Once the screw is positioned at a desired location on the bone surface, the screw is first rotated to insert the tip section of the screw a first distance into the bone. In particular, this first rotating causes a cutter on the tip section to remove a portion of the bone and create a pilot hole in the bone in which the screw tip is seated. Once the screw tip is seated, a second rotating step is performed to initiate insertion of a retention thread on the screw into the bone and advance the screw a second distance into the bone. During both rotating steps, the bone matter removed by the tip section cutter is deposited into a recessed channel in the screw tip from where it is then expelled between two successive coils of the thread. As will be appreciated, this expelled bone matter may then be forced upwards towards the bone surface as the screw is inserted. The step of positioning the screw tip may include a surgeon visually placing the point of the screw as near as possible to the center of an implantable device aperture. As will be appreciated, once the position is chosen, an axial pressure may be applied to the screw to press the screw into contact with the bone. Depending on the sharpness of the point as well as the hardness of the bone surface, this axial pressure may begin insertion of the screw tip into the bone prior to the first rotating step. Preferably, this downward axial pressure is continually applied to the screw during both the first and second rotating steps to aid in the screw's insertion into the bone.

When utilizing the bone screw to affix an implantable device to a bone, the step of advancing the screw a second distance into the bone may further comprise seating a head section of the screw into a countersunk bracket aperture. In this regard, care may be taken to insert the screw a second distance into the bone such that the bracket is firmly seated against the bone without damaging the bone structure. Preferably, the first and second rotating steps will require no more that a combined total of four to four and a half rotations to fully insert the screw into the bone and secure a bracket to the bone's surface.

According to another aspect of the present invention, a bone screw is provided that contains a head section having upper and lower surfaces, and a body section extending away from the lower surface of the head section and terminating in a point. The screw also contains a drive slot formed into the upper surface of the head section that extends across the width of the generally circular screw head, wherein each end of the slot passes through the upper surface of the head section to the lower surface of the head section to provide opposing contact surfaces for receipt within a correspondingly-shaped driver tool. In addition, the drive slot may have a width that allows it to form an interference fit with the driver tool.

The distance between the contact surface defined by the ends of the slot is less than that of the outside diameter of the head section. This allows an appropriately shaped drive tool (e.g., U-shaped) to both be received within the slot as well as extend through each end of the drive slot from the top surface to the lower surface such that the driver tool also receives the contact surfaces. In this regard, an appropriately sized and shaped driver tool may slidably fit over two or more of these contact surfaces and align the centerline axis of the screw with the centerline axis of the driver tool allowing aligned rotation of the screw with the driver tool during screw insertion. Furthermore, these contact surface may be formed to provide an interference fit within the driver tool. In this regard, the driver slot may provide dual interference fits with a driver tool while simultaneously providing alignment with the driver tool. As will be appreciated, this provides a secure “hands free” attachment of the screw to the driver tool prior to and during insertion of the screw into a bone.

### BRIEF DESRCIPTION OF THE DRAWINGS

FIG. 1 is a perspective view of one embodiment of the bone screw;

FIG. 2 is a side view of the bone screw of FIG. 1;

FIG. 3 is a plan side view of the bone screw of FIG. 1 in which the helical retaining tread has been removed for illustrative purposes;

FIG. 4 is an alternate side view of the bone screw of FIG. 1;

FIG. 5 is a cross-sectional view of the bone screw of FIG. 4 taken along section line A-A.

FIG. 6 is an end view of the bone screw of FIG. 1; and

FIG. 7 is a close-up view of the bone screw of FIG. 1 being inserted into a bone.

FIGS. 8a and 8b show one embodiment of a driver recess that may be utilized with the bone screw of FIG. 1;

FIGS. 9a-9d show a second embodiment of a driver recess that may be utilized with the bone screw of FIG. 1;

FIGS. 10a-10d show a driver bit that may be utilized with the driver recess shown in FIGS. 9a-9d; and

FIG. 11 shows a side view of the driver bit of FIGS. 10a-10d engaging the bone screw of FIG. 1 that contains a drive recess as shown on FIGS. 9a-9c.

### DETAILED DESCRIPTION

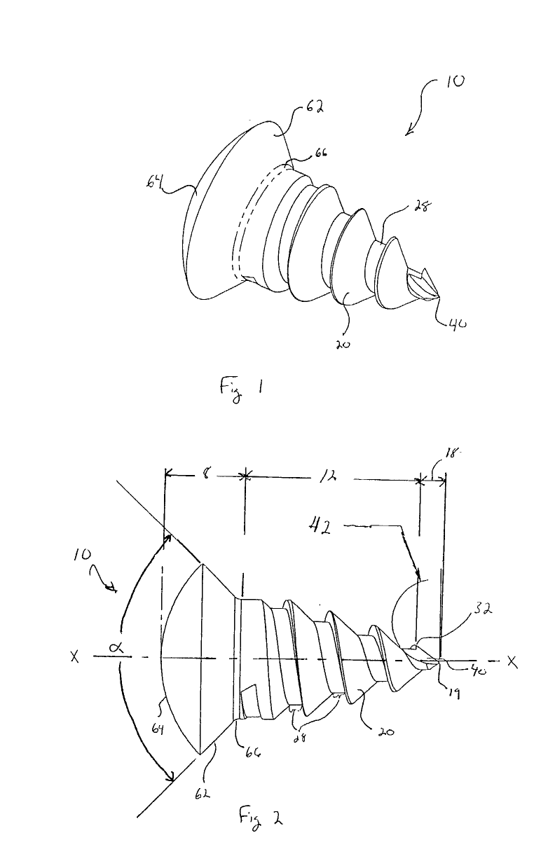

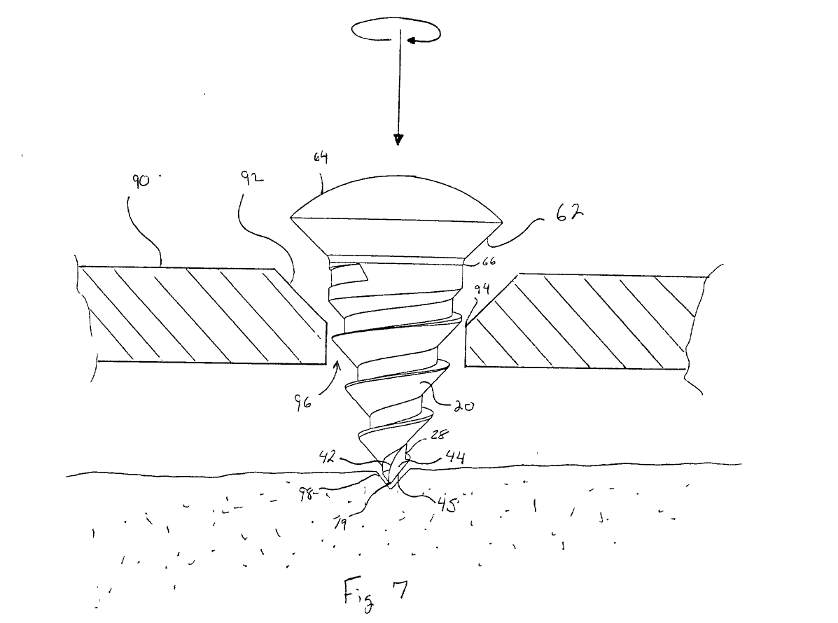

The present invention will now be described in relation to the accompanying drawings which at least partially assist in illustrating its various pertinent features. In FIG. 1, a perspective view of the bone screw 10 of the present invention is shown. Generally, the bone screw 10 comprises three sections: a head section 8, a body section 12 and a tip section 18 (see FIG. 2). Formed along the length of the body region's outside surface and extending from the tip section 18 to the head section 60 is a continuously expanding helical thread 20, as will be more fully discussed herein. Additionally, the tip section 18 of the screw 10 contains a recessed cutting flute 40 that enables the screw 10 to be self-drilling and enables the helical thread 20 to initially “bite” into a bone to allow the screw 10 to be self-tapping. The head section 8 generally comprises an angled lower flank surface 62 having a frusto-conical configuration. This lower flank surface 62 has an included angle α of 90°, creating a circular 45° flank surface 62 with respect to the screw's centerline axis X-X. This lower flank surface 62 is designed to be received within a countersunk recess within a prosthesis bracket in order to fasten that bracket to a bone surface (see FIG. 7). The head section 8 also contains a semi-circular upper surface 64 into which a drive recess (not shown) is formed for receiving a turning force to insert the bone screw 10 into a patient's bone.

The screw may be made of any material that provides the desired mechanical properties and is bio-compatible. A mechanical property of particular concern is a material's long term fatigue resistance, as the screws are intended for permanent use and long term fatigue may result in the degradation of the screw 10 over time, necessitating its replacement. Titanium and titanium alloys have been found to be particularly well-suited for bio-applications due to their long term fatigue resistance and bio-compatibility. In this regard, the bone screw 10 may be constructed of Grade 6 commercially pure titanium (Ti-6Al-4V E.L.I) or other machinable titanium grades. Additionally, some stainless steels, such as high nickel content stainless steels, may be used as well.

The illustrated embodiment of the bone screw 10 is primarily designed for attachment of implantable devices to cranial bone surfaces. In one particular application, one or more of the bone screws 10 are utilized to attach an implantable hearing aid system, which generally entails the subcutaneous positioning of various componentry on or within a patient's skull, at locations proximal to the mastoid process of the cranium's temporal bone. Such componentry may include, inter alia, a receiver for receiving transcutaneous RF and/or acoustic signals and an interconnected processor to provide processed signals. Additionally, some form of actuator may be employed to utilize the processed signals to stimulate the ossicular chain and/or tympanic membrane within the middle ear of a patient. The bone screws 10 may be utilized to attach each of these components to the patient's skull or attach an associated retention bracket to the skull to which the various components may be attached. However, the illustrated screw 10 and variations thereof may also be utilized for other surgical applications.

The overall length of the bone screw as shown is not greater than about 4 mm, which coincides with the average minimum thickness of an adult cranial bone. Further, as noted, the bone head section 8 of the screw 10 contains a frusto-conical lower flank surface 62 that is receivable in a countersunk recess and aperture of a retention bracket. Therefore, the overall length of the screw 10 actually inserted into a patient's cranial bone is generally not greater than about 3.5 mm. However, it will be appreciated that the basic design of the bone screw 10 as described herein may be altered from these dimensions for use in other bone applications. Regardless of the overall length of the bone screw 10, it is designed to lodge within a patient's bone and provide a secure attachment for a implantable device without the screw 10 necessarily passing entirely through the bone. In this regard, the screw 10 is designed to provide enhanced gripping force over a short screw geometry, allowing secure thin bone anchoring.

To provide enhanced gripping force over a short screw geometry, the screw body section 12 is designed having a helical thread 20 that constantly bites into previously undisturbed bone (i.e., creates original bone-to-screw contact) along a majority of the length of the thread 20 as the screw 10 is inserted. In this regard, at least one dimension of the helical thread 20 is expanding along substantially the entire length of the thread 20 between the beginning point of the thread 20 near the tip section 18 to the termination point of the thread 20 near the head section 8. This expansion in at least one dimension ensures that most of the helical thread 20 passes through bone that has not been precut by a previous like-sized portion of the helical thread 20.

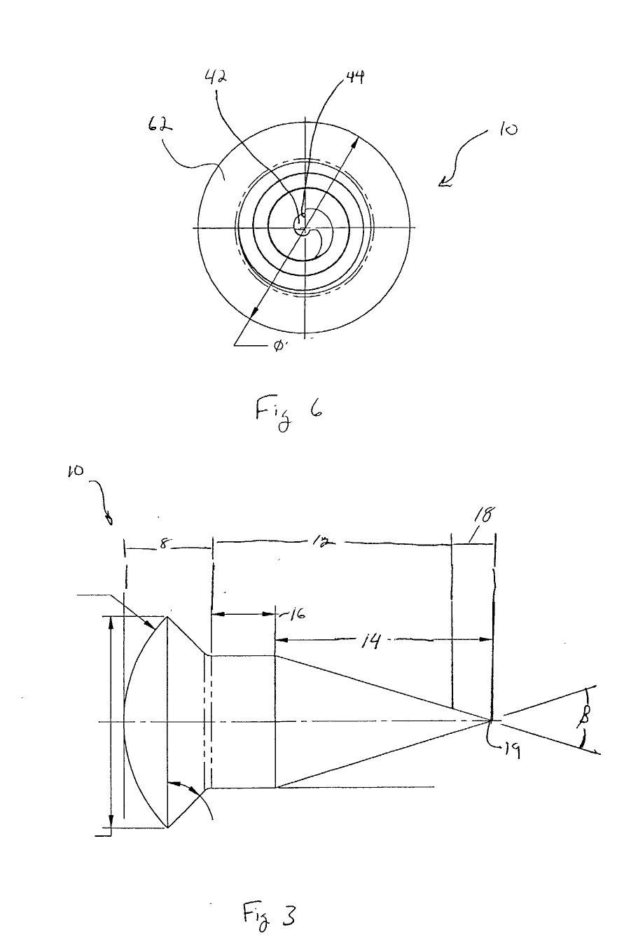

To allow the thread to expand in at least one dimension, the body section 12 of the screw 10 is generally tapered. FIG. 3 shows a plan side view of the bone screw 10, wherein the helical thread 20 has been removed for illustrative purposes. As shown, the body section 12 comprises a first tapered section 14 and a second alignment shank section 16. The body's tapered section 14 forms a cone between the point 19 of the screw 10 and the alignment shank section 16 and forms a majority of the overall length of the screw 10. As shown, the tapered section incorporates the tip region 18 and has an inclusive angle β of about 32.6°, however, tapered sections having smaller or larger included angles may also be utilized. This inclusive angle β describes the outside surface of the tapered section 14 exclusive of the helical thread 20 formed thereon. That is, the surface of the tapered section 14 having the inclusive angle of 32.6° forms a root surface 28 between successive coils of the helical thread 20 (see FIG. 2), as will be discussed herein. Further, it will be appreciated that the included angle β determines the overall length of the tapered section 14 for a given outside diameter of the shank section 16. That is, a smaller included angle β will produce a longer tapered section 14. However, a smaller included angle β will also define a slimmer cone (i.e., tapered section) producing screw 10 having less internal structure for withstanding insertion into a bone; therefore, the included angle β will generally be at least 20°. In any case, the tapered section 14 allows an outside diameter of the helical thread 20, as measured from a centerline axis X-X of the screw 10, to expand over the length of this tapered section 14. Accordingly, expansion of the thread 20 in the tapered section 14 ensures that no subsequent portion of the helical thread 20 passes through bone that has been precut by a previous like-sized portion of the helical thread 20.

In contrast to the tapered section 14, the alignment shank section 16 has a uniform outside diameter as measured from the central axis X-X of the screw 10. The main purpose of this constant diameter alignment shank section 16 is to center the screw 10 in a like-sized aperture within an implantable device such as a bone plate 90 (see FIG. 7). Typically, any such aperture will have a diameter substantially identical to the outside diameter of the alignment shank 16 and will be beveled such that the flank surface 62 of the head section 8 seats within the bevel 92. That is, the bone screw 10 is designed to fit snugly within an appropriately sized and countersunk bracket plate aperture 96. As will be appreciated, the necessity of the alignment shank 16 having a uniform outside diameter to matingly fit within a bracket plate aperture 90 prevents the outside diameter of the helical thread 20 from expanding in the alignment shank section 16 of the screw body. Therefore, if continued expansion of the thread 20 is desired to provide for original thread-to-bone contact within the alignment shank section 16, (a portion of which may also be inserted into the bone) a thread dimension other than the outside diameter must expand over the alignment shank section.

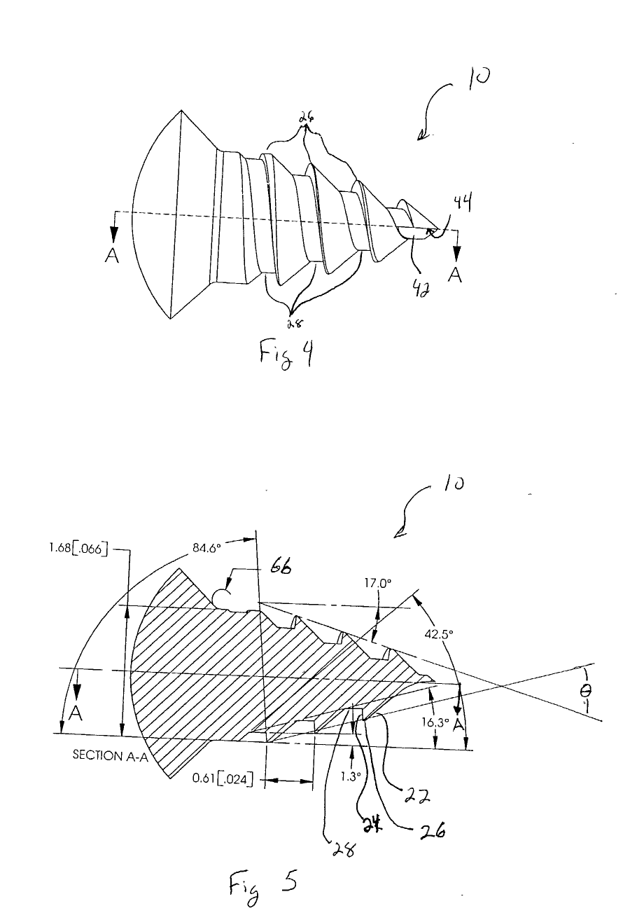

Referring to FIG. 5 which is a cross-sectional view taken along section line A-A of FIG. 4, it will be noted that the helical thread 20 generally includes four elements: a leading flank 22, a trailing flank 24, a crest surface 26 disposed between the flanks 22 and 24 and a root surface 28 separating successive coils of the helical thread 20. The cross-sectional view of FIG. 5 also best illustrates the continual expansion of one or more of the thread dimensions. As shown, within the tapered section 14 of the body 12, the outside diameter of the thread 20 or “crest” 26 of successive helical coils forms an expanding conical spiral having an included angle θ of 34°. Accordingly, the outside diameter of the thread crest 26, as measured from the screw's centerline axis X-X, continues to increase in diameter throughout the entire length of the tapered section 14 of the screw 10 until the thread 20 reaches the alignment shank section 16, which has the constant outside diameter. In this section 16, the crest diameter, as measured from the centerline axis X-X, becomes a constant. However, the root surface 28 of the thread 20 continues to expand in diameter (as measured from the screw's centerline axis X-X) throughout the alignment shank section 16. Due to the continual expansion of the root surface 28, the overall height of the thread 20 decreases in the shank section 16. Accordingly, the width of the crest 26 increases throughout the constant diameter shank section 16 (see FIG. 4). In this regard, two thread dimensions, the crest width and root surface diameter, as measured from the centerline axis X-X, continue to expand throughout the constant diameter alignment shank section 16. Again, expansion of these thread dimensions ensures that no portion of the helical thread 20 passes through bone that has been precut by a previous like-sized portion of the helical thread 20. In order for the root surface 28 and crest width 26 to expand throughout the shank section 16, the shank section 16 cannot be longer than about 1 to 1.5 times the thread pitch (i.e., the distance between successive crests) of the screw 10. This design, wherein at least one dimension of the helical thread 20 is continuously expanding along the entire length of the thread (i.e., root surface diameter, outside crest diameter, and/or crest width), allows the bone screw 10 to continuously dig into the bone during insertion and provide increased original screw-to-bone contact that increases the gripping force provided by the screw 10.

The cross-sectional shape of the helical thread has also been formed to provide increased holding force. As shown in FIG. 5, the helical thread 20 contains a leading flank 22 and a trailing flank 24 along the entire length of the screw 10. The leading flank 22 is formed at an angle of about 42.5° as measured from the centerline axis X-X. In contrast, the trailing flank 24 contains an angle of approximately 84.6°, as measured from the centerline axis X-X. The angle for the leading flank 22 was chosen primarily to maximize its surface area and to allow the screw thread 20 to be machined on the body section 12 utilizing a single tool. However, the angle of the trailing flank 24 was specifically chosen to be as near as perpendicular to the central axis as practicable to provide additional gripping force and facilitate in removal of any bone fragments cut by the cutting flute 40. In this regard, the near perpendicular trailing flank 24 creates a nearly square platform edge having increased resistance to axial extraction forces applied along the centerline axis X-X of the screw 10. Further, bone fragments created by the cutting flute 40 during insertion of the screw 10 ride atop this square platform edge during screw insertion. As will be appreciated, in screws that utilize a more a more tapered trailing flank, bone fragments are more likely to slide towards the thread crest where they may wedge between the crest and the bone. In cranial applications where the bone is formed from a series laminated bone layers, these wedged fragments may cause undue pressure between successive layers and thereby compromise the screw's gripping force.

The continually expanding helical thread design provides an additional benefit, namely, easy removal of the screw 10. As shown, the helical thread 20 has a constant pitch (i.e., distance between successive coils along the length of the body 12) that allows the screw 10 to be seated within about four to four and one-half turns. However, due to the tapered design and continuous expansion of the helical thread 20, the bone screw 10 may be removed at any point by turning the screw 10 about one-half turn backward. In this regard, turning the screw 10 about one-half turn backward releases all the helical threads from the groove that they have cut into the patient's bone and allows the smaller preceding portions of the thread 20 to be extracted therethrough without damaging the thread tapped into the bone. Accordingly, the bone screw 10 may be partially inserted during a surgical procedure, extracted and reinserted into the tapped screw hole and retightened, without affecting the gripping force of the screw 10.

Another benefit of the continually expanding thread 20 is that the bone screw 10 is resistant to stripping. As will be appreciated, most screws utilize a short tip section that begins a tap thread within the bone for all subsequent like-sized threads on a constant diameter body. If this first tap thread is stripped during screw insertion, the screw insertion position is ruined. Therefore, by utilizing a continually expanding thread, no portion of the bone may be stripped by a preceding thread such that the current portion of the thread cannot continue to dig into or “bite” into the bone. The only time the threads tapped into a bone may be irretrievably stripped by the bone screw 10 is when the entire bone screw 10 is fully seated within the target bone and a surgeon continues to turn the screw 10.

To prevent stripping threads within the bone when the screw 10 is fully seated, the head portion 8 of the screw 10 contains a rounded contact surface 66 where the head's lower flank 62 meets the alignment shank 16 (see FIGS. 2 and 5). This rounded contact surface 66 is designed to provide an additional resistance torque to the turning of the bone screw 10 when this rounded contact surface 66 contacts a squared mating surface 94 within a bracket (see FIG. 7). That is, the rounded contact surface 66 will contact the mating surface of a bracket 90 just prior to the screw's lower flank 62 fully seating within a countersunk hole in that bracket 90. Upon the rounded contact portion 66 contacting this surface, a surgeon inserting the screw 10 will feel an increase in resistance and therefore realize that the screw 10 is fully set prior to stripping the threads within the bone.

The bone screw 10, as noted, also contains a tip section 18 incorporating a cutting flute 40. As shown in FIGS. 1, 2, 4 and 6, the cutting flute is formed from a substantially planar surface 44 and a arcuate surface 42 recessed into the screw 10. The arcuate surface 42 is generally an arcuate recess cut into the tip section 18 that is best shown in the side view of FIG. 2A side view along the planer surface 44 is shown in FIG. 4. The planar surface 44 and arcuate shaped surface 42 form a substantially right angle recess, wherein the crux of this right angle meets directly upon the centerline axis X-X of the screw tip 19, as shown by the end view of FIG. 6. The centering of the cutting flute 40 with the centerline axis X-X at the tip 19 provides an alignment benefit during insertion of the bone screw 10. Particularly, the centered cutting flute 40 allows the bone screw 10 to dig into a bone almost exactly where the screw 10 is placed on the bone. As will be appreciated, bone screws that contain cutting flutes that are somewhat offset of the centerline of the bone screw tend to wobble as they are inserted and, therefore, are apt to move slightly during insertion. That is, these screws may not screw in exactly where they are placed. This can be problematic in delicate surgical procedures, for example, where two or more bone screws are utilized to hold a bracket wherein movement of one bone screw may misalign either the bracket or the alignment of the other screw with the apertures of that bracket.

Referring again to FIG. 2, it will be noted that the recessed cutting flute 40 intersects the cross section of the helical thread 20 in only a single position. That is, most of the helical thread 20 is formed on the screw body 12 after the recessed cutting flute 40. In this regard, the recessed cutting flute's planar surface 44 intersects the very beginning portion of the helical thread 20, creating a small barb 32 at the beginning of the helical thread 20. This barb 32 has been designed to have a minimum cross-sectional size while still allowing the helical thread 20 to begin tapping into the bone as the screw 10 is turned. It will be appreciated that by minimizing the size of the barb 32, less bone is removed while the screw 10 is inserted into the bone, allowing for a tighter grip between the bone and the screw 10.

Though discussed above in relation to utilizing the bone screw 10 to affix prosthetic devices to a patient's bone, the bone screw of the present invention may also be utilized as a bone anchor. In this embodiment, two or more screws may be inserted within two or more bone fragments and then interconnected using, for example, wires. In this regard, the head section's lower flank surface 62 may be formed with a greater included angle α (i.e., 120°-180°) such that a wire or a suture may be wound about the alignment shank 16 and entrapped between the surface of a patient's bone and the flank surface 62. As will be appreciated, in this embodiment only the tapered section 14 of the screw's body 12 will be screwed into a bone. Alternatively, the screw's head section 8 may be formed containing some sort of aperture through which a wire/suture may pass.

Referring to FIGS. 2, 4 and 7, insertion of the bone screw 10 into a bone is described. Initially, the bone screw 10 is positioned on a bone's surface in a desired position (see FIG. 7). This generally entails a surgeon visually placing the screw tip 19 as near as possible to the center of a prosthetic bracket aperture. Upon selective placement of the screw 10 within a bracket aperture, pressure is applied along the screw's centerline axis X-X while the screw 10 is rotated in a clockwise fashion as shown by the arrows in FIG. 7. As will be appreciated, the step of rotating is performed by inserting the end of a driver tool into a correspondingly configured drive recess, as will be discussed herein, within the semi-circular upper surface 64 of the bone screw head section 8. The particular configuration of this drive recess is incidental to the operation of the bone screw 10. However, it is preferable that the driver tool and drive recess correspondingly mate to allow the bone screw 10 to be retained on the end of the driver tool for positioning during surgical procedures. In this regard, some sort of compression fit between the end of the driver and the driver recess may be utilized or, in the case of ferromagnetic screws, a magnetic tipped driver tool may be utilized. Regardless of the driver tool and drive recess utilized, once positioned, the screw is rotated in a clockwise manner to insert the screw 10 into a target bone.

Initially, the screw tip 19 is inserted a first distance into the bone. As noted above and shown in FIG. 7, the tip section 18 contains a recessed cutting flute 40 having a planer surface 44 and an arcuate surface 42 that form a right angle recess into the screw 10. A first end of this right angle recess has a crux that meets directly upon the screw's centerline axis X-X at the screw tip 19. The planer surface 44 of the flute 40 is aligned with the centerline axis X-X of the screw 10 along its entire length (see FIG. 4), creating a flat surface facing towards the direction of screw 10 rotation. In this regard, the planer surface 44 forms a cutting edge 45 operable to remove a portion of bone matter as the screw 10 is rotated clockwise. In contrast, the arcuate surface 42 begins aligned with the centerline axis X-X at the screw tip 19 and arcs upward along its length until it terminates at the root surface 28 between the first and second thread coils. The bone matter removed by the cutting edge 45 is received within the recessed cutting flute 40 as the screw 10 is rotated. Accordingly, as additional bone matter is received near the tip of the recessed flute 40, earlier removed bone matter is expelled out of the rear of the flute 40 between the thread coils. That is, the arcuate surface 44 is configured to direct the removed bone matter out of the recessed cutting flute 40 where it is deposited between the first and second coils of the screw thread 20. This expelled bone matter then is pushed upwards along the trailing flank 24 of the thread 20 towards the bone's surface as the screw 10 is inserted.

During initial insertion of the screw 10, the recessed cutting flute 40 “drills” a circular hole 98 into which the tip section 18 is seated prior to the thread 20 beginning to tap into the bone. Once seated within the circular hole, the tip section 18 acts as a spindle within the bone that holds the screw 10 at its initial placement spot even when the thread's burr 32, which is somewhat offset of the screw's centerline axis X-X, begins to be inserted into the bone. That is, the initial screw 10 insertion a first distance into the bone (i.e., the length of the tip section) without insertion of the thread 20 allows the screw 10 to be affixed to its desired position, which prevents the screw 10 from wobbling during the remainder of its insertion which may result in screw movement and/or misalignment. Accordingly, after tip insertion, the screw 10 is inserted into the bone a second distance in which the continuously expanding thread 20 taps into the bone. This second distance insertion draws the aperture bracket into contact with the bone's surface and seats the lower flank surface 62 of the screw head section 8 into the correspondingly shaped recess bevel 92.

FIGS. 8a and 8b show one embodiment of a driver recess 100 utilized for applying an insertion torque to the bone screw 10. As shown in FIG. 8a, a driver recess 100 is formed into the top surface 64 of the head section 8 of the screw 10. In this embodiment, the driver recess 100 is a hexagonal six-sided recess centered with the centerline axis X-X of the screw 10 and centered within the top surface 64 of the head section 8 (see FIG. 8b). As will be appreciated, the hexagonal driver recess 100 allows a hexagonal driver bit (not shown) to apply a turning force or torque to the screw 10. In order for the screw 10 to be maintained on the end of the hexagonal driver bit, the driver bit will form an interference fit with the hexagonal driver recess 100. That is, at least two opposing sides of the hexagonal recess 100 will be spaced a smaller distance from one another than the corresponding outside width of opposing surfaces of the driver bit. In this regard, an interference fit will be formed between the driver bit and the driver recess 100, which will allow the screw 10 to be maintained on the end of the driver bit for hands-free insertion.

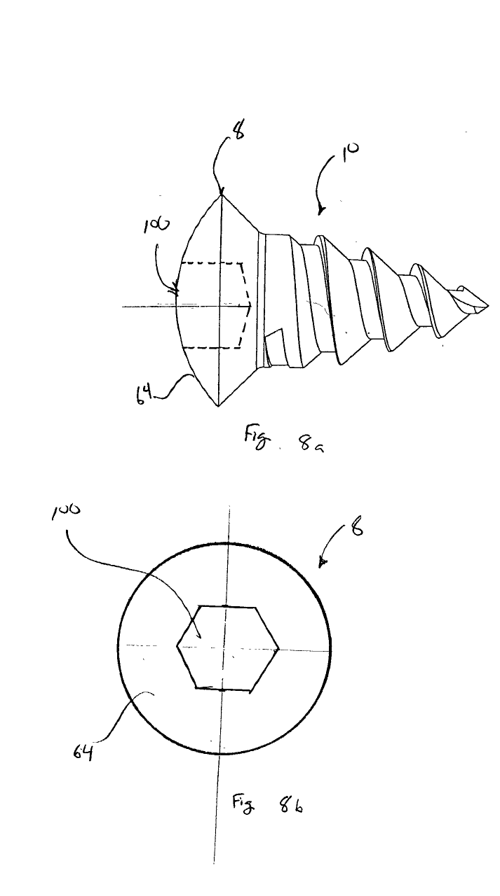

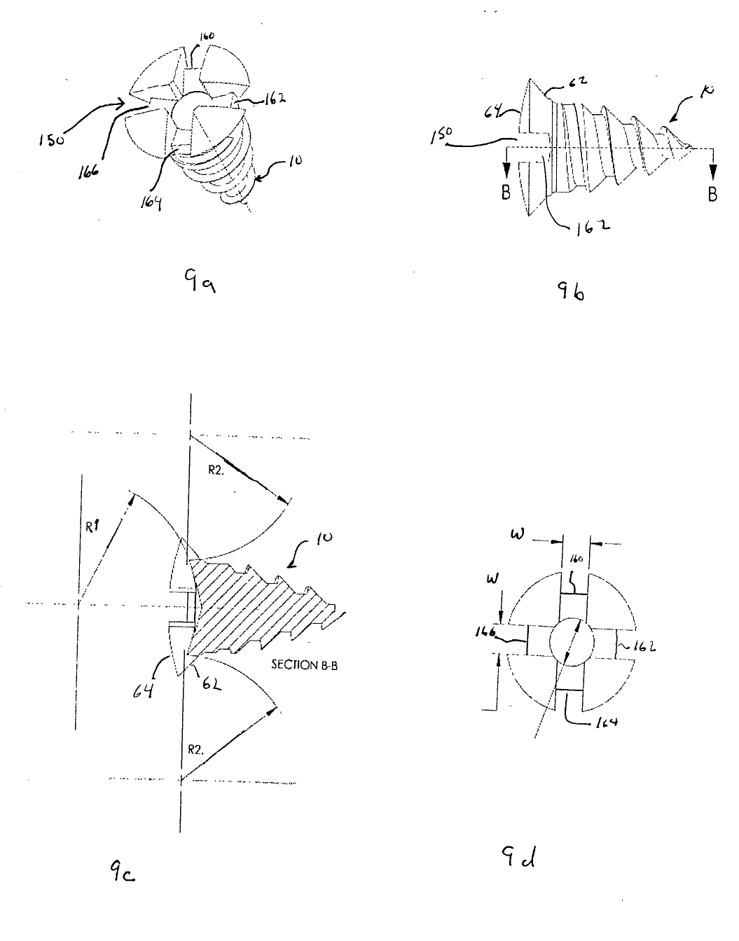

FIGS. 9a-9d show a second embodiment of a drive recess 150 that may be utilized with the bone screw 10. In this second embodiment, the drive recess 150 is formed as a cruciform slot through both the top surface 64 and flank surface 62 of the head section 8 of the screw 10. As shown in FIG. 9b (a cross-section view of FIG. 9b), the cruciform slot is formed by cutting a first circular groove into the top surface 64 of the screw 10. This circular groove has a first radius of R1. Once the two slots that make up the drive recess 150 are cut into the top surface 64, the ends of each slot are counter-cut about a second radius R2. That is, the material of the screw head 8 between the upper surface 64 and lower frusto-conical flank surface 62 is removed (see FIGS. 9a and 9c). The resulting cruciform drive recess 150 contains four contact surfaces 160-166 that collectively define a centering “drive lug.” Furthermore, all the slots are cut into the head 8 of the screw 10, having a width (W) as shown in FIG. 9d.

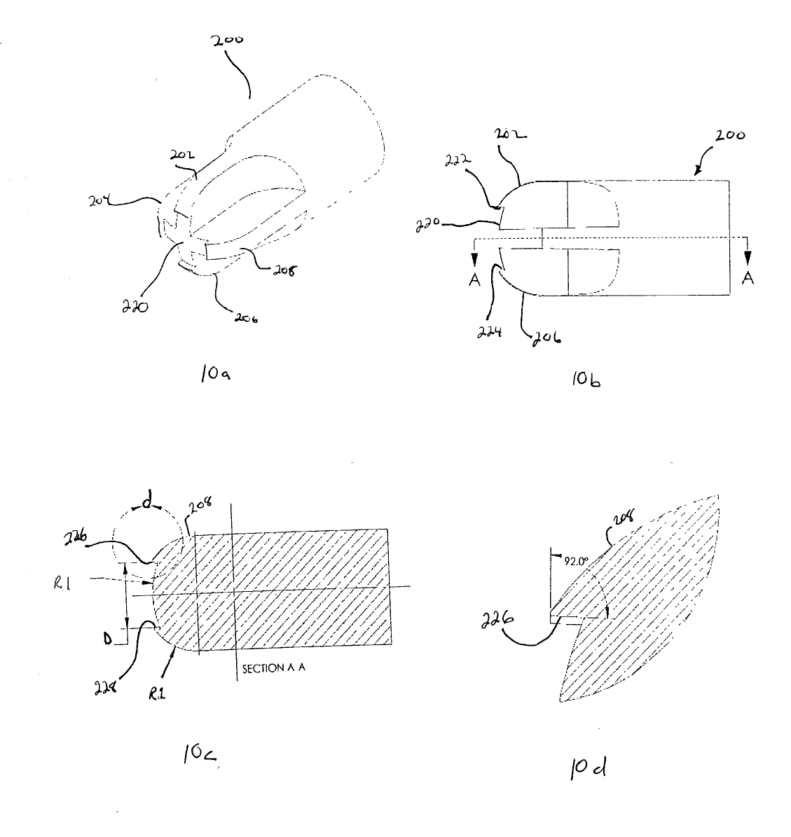

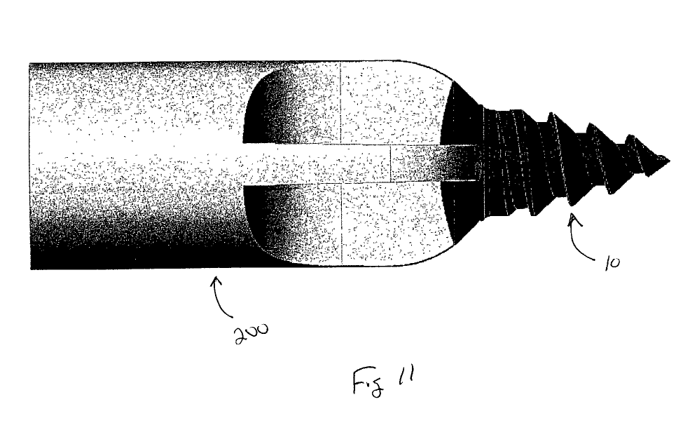

FIGS. 10a-10d illustrate a double interference fit drive bit for use with the drive recess described above. As shown, the drive bit 200 contains four blades 202-208 sized to be received within the cruciformed drive slot 150. Each of these blades 202-208 is sized to be slightly wider than the width (W) of the cruciform drive slots. In this regard, the outside surfaces of each blade 202-208 will form an interference fit with the slightly more narrow drive slots. Furthermore, the drive bit 200 contains a “socket” surface 220 recessed into the tip of the drive bit 200 and the four blades 202-208. FIG. 10b shows a side view of the drive bit 200 and two abutments 222, 224 that are formed into blades 202 and 206 by the socket surface 220. FIG. 10c shows a cross-sectional view taken along section lines 8a of FIG. 10b and shows the two abutments 226 and 228 formed into blades 208 and 204, respectively. As shown, the two abutments 226 and 228 are spaced a distance (D) apart. This distance (D) is made to correspond with the distance between the opposing contact surfaces 160-166 that collectively define the drive lug of the cruciform driver recess 150. Again, this distance (D) may be slightly less than the distance between the opposing contact surfaces (e.g., 160 and 164), allowing for a second contact fit between the driver bit 200 and the screw 10. In this regard, as shown in FIG. 10d abutment 226 may have an angle slightly greater than a right angle to account for the radius cut (i.e. R2) of the contact surfaces 160-160 and thereby provides a better interference fit. Further, the socket surface 220 is a hemispherical surface to mate with the bottom of the cruciform slots (i.e. has a radius of R1). As will be appreciated, by utilizing the socket surface 220 abutments 222-228 and the contact surfaces 160-166 on the screw 10, the screw 10 will necessarily be directly centered on the drive bit 200 prior to insertion into a target bone. Furthermore, due to the dual interference fit provided by the abutments 222-228 slidably engaging the contact surfaces 160-166 as well as the blades 202-208 being slidably received within the drive slots, the screw 10 is securely fastened to the drive bit 200 to allow “hands-free” insertion of the screw 10. That is, the screw 10 will maintain a fixed positional relationship on the end of the driver bit 200 without being manually held in contact thereto as shown in FIG. 11. FIG. 11 shows a side view of the driver bit 200 engaged into a screw 10 containing the cruciform drive recess 150. It will be noted that when the “lug” is secured within the socket surface 220, the outside edge of each blade 202-208 has a diameter slightly less than the maximum screw diameter as defined by the outside perimeter of the screw head 8. In this regard, the blades 202-208 of the driver bit 200 will not engage a beveled surface of a prosthesis bracket during insertion of the screw 10.

The foregoing description of the present invention has been presented for purposes of illustration and description. Furthermore, the description is not intended to limit the invention to the form disclosed herein. Consequently, variations, adaptations, modifications, and skill and knowledge of the relevant art, are within the scope of the present invention as determined by the claims that follow.

What is claimed is:

1. A self-drilling, self-tapping bone screw, said bone screw comprising:
a screw head;
a tip section having a cutter for initiating insertion of said screw into a bone;
a body section extending between said screw head and said tip section; and
a helical thread formed on the outside surface of said body section, wherein an outside diameter of said thread, as measured from a centerline axis of the screw, expands over a majority of the length of said helical thread allowing said majority of said thread to continuously tap into a bone during insertion of said screw.

2. The bone screw of claim 1, wherein said outside diameter of said thread expands over at least seventy-five percent of the length of said thread.

3. The bone screw of claim 1, wherein said outside diameter of said thread expands over a first portion of the length of said body section and a second dimension of said thread expands along at least a second portion of said body section.

4. The bone screw of claim 3, wherein said second dimension of said thread is selected from a group of dimensions consisting of:
an outside diameter of a root surface between successive coils of said helical thread, wherein said outside root diameter is measured from the centerline axis of said bone screw;
a width associated with the top surface of said helical thread;
an angle of the leading flank of said thread as measured from the centerline axis; and
an angle of the trailing flank of said thread as measured from the centerline axis.

5. The bone screw of claim 4, wherein at least one of said outside diameter of said thread and said second dimension of said thread are expanding over the entire length of said thread.

6. The bone screw of claim 1, wherein a majority of said body section is tapered between said tip section and said screw head.

7. The bone screw of claim 6, wherein said tapered section defines a cone having an included angle of less than about 45°.

8. The bone screw of claim 1, wherein said helical thread contains a trailing flank surface that forms an acute angle, measured from the centerline axis of said bone screw, of at least about 85°.

9. The bone screw of claim 1, wherein said cutter comprises a flute defined by two surfaces recessed into said tip section.

10. The bone screw of claim 9, wherein said two surfaces recessed into said tip section intersect at the centerline axis at an end point of said tip section allowing said flute to cut into a bone substantially where said end point is positioned on the bone.

12. The bone screw of claim 9, wherein at least one of said surfaces comprises an arcuate surface configured to direct removed bone matter between successive coils of said helical thread during screw insertion.

13. The bone screw of claim 1, wherein said tip section and said body section are in combination no greater than about 4 mm in length.

14. The bone screw of claim 1, wherein said screw head further comprises a drive recess for receiving a turning force to insert said bone screw into a bone.

15. The bone screw of claim 14, wherein said drive recess comprises a hexagonal recess centered within said screw head.

16. The bone screw of claim 14, wherein said drive recess comprises a cruciform drive slot.

17. A self-drilling, self-tapping bone screw, said bone screw comprising:
a screw head;
a generally tapered body section extending from said screw head and terminating in a point, said body section being tapered from a first diameter beginning at said point to a second diameter ending near said screw head, wherein said second diameter is greater than said first diameter;
a helical thread formed on the surface of at least a portion of said tapered body section, wherein said thread has an expanding outside diameter along said portion of said tapered body; and
a cutting flute associated with said point and extending along a portion of said tapered body section.

18. The bone screw of claim 17, wherein said outside surface of said tapered body section, exclusive of said retaining thread, comprises a cone shape.

19. The bone screw of claim 18, wherein said cone shape has an included angle of not greater than about 45°.

20. The bone screw of claim 18, wherein the distance between said point and said tapered body section having said second diameter comprises more than about seventy-five percent of the overall length of said tapered body section.

21. The bone screw of claim 20, wherein at least a second dimension of said helical thread formed on the surface of said cone shaped body expands along at least a second portion of said tapered body.

22. The bone screw of claim 21, wherein said second dimension comprises at least one of:
a root surface diameter measured from the centerline axis of said bone screw, wherein said root surface is the surface between successive coils of said helical thread; and
a crest width of said helical thread;
an angle of the leading flank of said thread as measured from the centerline axis; and
an angle of the trailing flank of said thread as measured from the centerline axis.

23. The bone screw of claim 20, wherein at least one dimension of said helical retaining thread expands along the entire length of said thread.

24. The bone screw of claim 23, wherein a first dimension of said helical retaining thread expands along a first portion of said body section and a second dimension of said helical retaining thread expands along a second portion of said body section.

25. The bone screw of claim 17, wherein said cutting flute comprises two surfaces recessed into said screw that intersect at the centerline axis of said screw at the end of said screw point, wherein said centered cutting flute allows said point to cut into a bone substantially where said point is positioned on the bone.

26. The bone screw of claim 17, wherein said screw head further comprises a drive recess for receiving a turning force to insert said bone screw into a bone.

27. A self-drilling, self-tapping bone anchor, said anchor comprising:
a head for receiving an insertion torque for inserting said anchor into a bone;
a body section extending from said head and terminating in a point, wherein said point includes a cutter for initiating insertion of said anchor into a bone;
a continuous helical thread formed on the outside surface of said body section, wherein at least one dimension of said continuous helical thread expands along a majority of the length of said thread allowing said majority of said thread to continuously dig into a bone during insertion of said anchor; and
a retention element associated with said head for selectively receiving a surgical securing device.

28. The bone anchor of claim 27, wherein said at least one expanding thread dimension comprises at least one of:
an outside thread diameter measured from the centerline axis of said bone screw;
a root surface diameter measured from the centerline axis of said bone screw, wherein said root surface is the surface between the helical coils of said helical retaining thread; and
a crest width of said helical retaining thread.

29. The bone anchor of claim 27, wherein said body section is tapered from a first diameter beginning at said point to a second diameter ending near said screw head, wherein said second diameter is greater than said first diameter.

30. The bone anchor of claim 27, wherein said retention element comprises a lip surface formed around said head section for entrapping said surgical securing device between said lip and a bone surface.

31. The bone anchor of claim 27, wherein said retention element comprises an aperture through said head section for receiving said surgical securing device.

32. A method of inserting a self-tapping self-drilling bone screw into a bone, said method comprising the steps:
positioning a tip of said bone screw at a desired location on a target bone surface;
first rotating said bone screw to advance said screw tip a first distance into the bone, wherein said tip removes a portion of bone matter to form a pilot hole at said desired location;
second rotating said bone screw while said tip is inserted in said pilot hole to initiate insertion of a screw thread into said bone and advance said screw a second distance into the bone; and
said bone matter removed by said tip being displaced through a recessed channel in said screw tip configured to expel said removed bone matter between two successive coils of said thread.

33. The method of claim 32, wherein said second rotating step taps a female screw thread into said bone, wherein at least one dimension of said female thread is expanding over a majority of its length.

34. The method of claim 32, wherein said positioning step further comprises inserting said bone screw through an aperture of an implantable device prior to positioning said tip on said bone.

35. The method of claim 34, wherein said advancing said screw a second distance secures said device to said bone surface.

36. The method of claim 34, wherein said advancing said screw a second distance secures a forward surface of a screw head of said screw to the top surface of said device.

37. The method of claim 32, wherein said first and second rotating steps further comprise applying an axial force to said screw to force said screw into contact with said bone.

38. The method of claim 32, wherein said first and second rotating steps seat fully said screw within a bone in no more than about four to four and a half rotations.

39. A self-drilling self tapping bone screw, comprising:
a head having an upper surface and a lower surface;
a body section extending from said lower surface and terminating in a point, wherein said point includes a cutter for initiating insertion of said bone screw into a bone;
a continuous helical thread formed on the outside surface of said body section; and
a drive slot formed into said upper surface extending across the entire width of said head, wherein at least a portion of each end of said slot pass through said upper surface to said lower surface forming opposing contact surfaces.

40. The bone screw of claim 39, further comprising a contact surface formed where each end of said drive slot passes through said upper surface to said lower surface.

41. The bone screw of claim 40, wherein said contact surfaces are sized to be slidably received within a driver and form a first interference fit with the driver.

42. The bone screw of claim 41, wherein said contact surfaces axially align a centerline of said screw and a centerline of the driver when said surfaces are slidably received within said driver 43. The bone screw of claim 39, wherein said drive slot is sized slidably receive a driver and form a second interference fit with the driver.
